(19) United States
(12) Patent Application Publication
(10) Pub. No.: US 2007/0148696 A1
(43) Pub. Date: Jun. 28, 2007

(54) Highly-selective tandem chemical sensor and detection method using same

(76) Inventors: Celine Fiorini-Debuisschert, Orsay (FR); Vesna Simic, Paris (FR); Olivier Vigneau, Gif-Sur-Yvette (FR); Pierre Le Barny, Orsay (FR)

Correspondence Address: LOWE HAUPTMAN GILMAN & BERNER, LLP, 1700 DIAGNOSTIC ROAD, SUITE 300, ALEXANDRIA, VA, 22314, US

(21) Appl. No.: 10/589,467

(22) PCT Filed: Feb. 9, 2005
(86) PCT No.: PCT/EP05/50573

§ 371 Date: Aug. 14, 2006

(30) Foreign Application Priority Data
Feb. 12, 2004 (FR) 04 01395

(51) Int. Cl. G01N 33/53

(52) U.S. Cl. 435/7.1; 436/86

(57) ABSTRACT

The invention relates to a chemical sensor intended for the detection of a type of molecule comprising a fluorescent material capable of forming a charge transfer complex with the type of molecule to be detected and means for measuring the variation in fluorescence of said material, characterized in that it additionally comprises a filter comprising a polymer material comprising “molecularly imprinted” cavities, the geometric and chemical configuration of which is defined so as to fix the type of molecule to be detected. The invention also relates to a detection method using the sensor according to the invention.

The field of the invention is that of chemical sensors and in particular sensors capable of detecting particularly dangerous molecules, such as explosives, drugs, and the like.

Generally, a chemical sensor comprises a sensitive layer brought into contact with a transducer which translates into a readily quantifiable signal the chemical signal generated following the interactions between the sensitive layer and the compound to be detected. An efficient chemical sensor must thus fulfill the following two conditions: be capable of easily creating interactions with the molecule to be detected and generating a readily observable signal.

A very large number of technological solutions in the field of gas detection are available today. However, there is still no system which combines high selectivity, very high sensitivity and very short response time for the detection of dangerous gases.

As regards the detection of explosives (aromatic nitro derivatives), there are mainly four types of sensors which are currently under development:
a sensor based on ion mobility measurements (IMS) which makes it possible to identify the molecules after they have been ionized and deflected under an electrical field (Ion Track's Itemiser, GE-Interlogix). Tests show that this sensor is effective for detecting compounds having a high vapor pressure but is ineffective for detecting TNT or DNT (Singh S., Singh M., Signal Processing, 2003, 83, 31-55)
a surface acoustic wave sensor (Naval Research Laboratory, Geo-Centers Inc., Nova Research Inc.)
distance detection of modification of fluorescence of particles which preconcentrate aromatic derivatives (Sandia National Laboratories)
an optical sensor based on the quenching of the fluorescence of a π-conjugated polymer and devoted to the detection of antipersonal mines (Nomadics Inc. and MIT) (Patents Swager T. M., EP 1 281 744, WO 02/16463, EP 1 263 887). The authors claim the detection of traces of TNT at concentrations as low as a few ppt (parts per trillion). The efficiency of the sensitive layer results from the effect of chemical amplification brought about by the presence of π-conjugated macromolecules.

Although this type of sensor, developed by Nomadics Inc., appears highly efficient, it is apparent that the selectivity is only partial since, a priori, numerous molecules of electron-deficient type can quench the fluorescence of the polymer and thus result in false alarms.

In the case of molecules of explosives, the potential interfering entities can be fragrances, some of which are represented below.

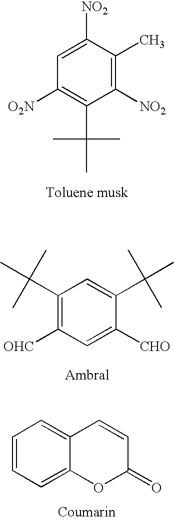

Nitrobenzene, a by-product of tobacco, can also distort the results detected.

This is why the present invention provides a highly selective chemical sensor in which the detection of molecular entities by a variation in fluorescence is combined with the prior selection of said entities by a chemical filter based on molecularly imprinted material.

Thus, in order to overcome the lack of selectivity of the sensors of the state of the art and to prevent any problem of “false alarm”, the invention provides a novel concept of sensor in which a material which will sort the molecules (filter) is combined with a fluorescent material which will act as sensitive layer.

It should also be noted that, in this way, the risks of saturation of the sensitive polymer as a result of the adsorption of interfering molecules is also limited.

More specifically, a subject matter of the invention is a chemical sensor intended for the detection of a type of molecule comprising a fluorescent material capable of forming a complex with the type of molecule to be detected and means for measuring the variation in fluorescence of said material, characterized in that it additionally comprises a filter comprising a polymer material comprising “molecularly imprinted” cavities, the geometric and chemical configuration of which is defined so as to fix solely the type of molecule to be detected.

Advantageously, the fluorescent material can be a polymer or an assembly of small molecules. The fluorescent polymer can be a polymer comprising a π-conjugated chain, for example of the following type 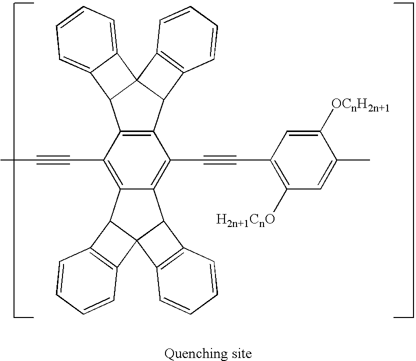
It can also be a polymer comprising side chains of the following type:
General Formula: 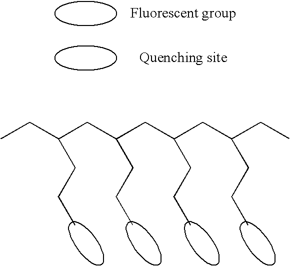
Examples: 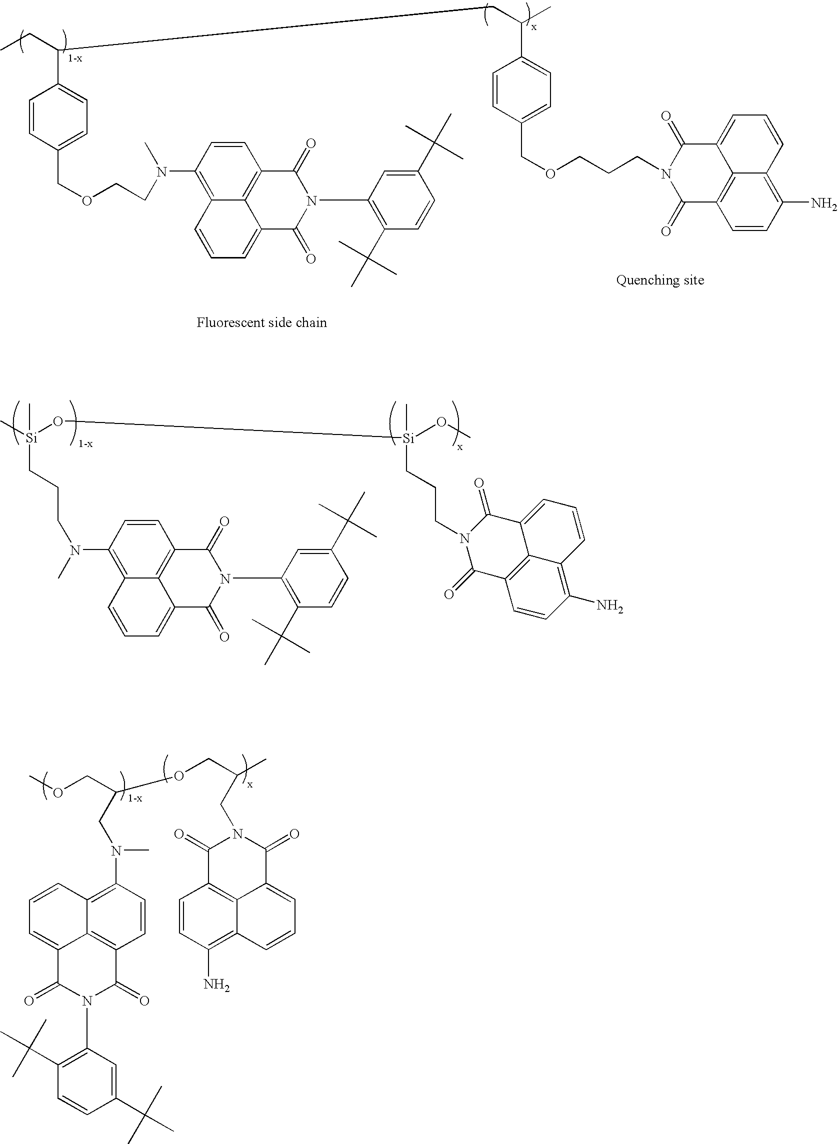
with x molar fraction. Preferably, the choice will be made of x<0.05.

Advantageously, the polymer material comprising “molecularly imprinted” cavities can be obtained from functional monomers capable of complexing the molecule to be detected, it being possible for the interactions to be of the hydrogen bond type, 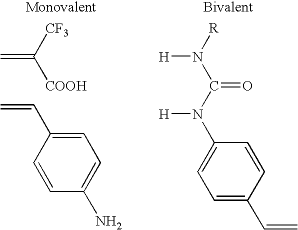
or of π-π interactions type 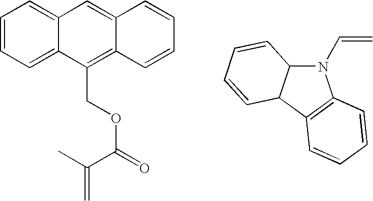
or of metal-ligand complexes type

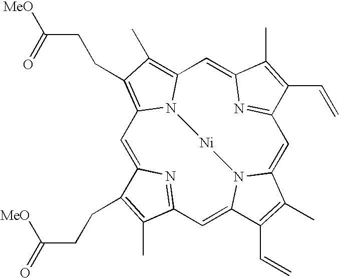

In the sensor according to the invention, the fluorescent material can be deposited as a thin layer at the surface of at least one first substrate.

The polymer material comprising “molecularly imprinted” cavities can be produced at the surface of a membrane or at the surface of microbeads so as to produce a maximum exchange surface area with the outside and so as to also make possible a response time (time for adsorption of the molecules to be detected) which is as short as possible. More specifically, it can be formed at the surface of a membrane or at the surface of microbeads held in a porous support positioned perpendicular to the charged stream or positioned parallel to the gas stream and arranged in a column of chromatography column type.

Advantageously, the sensor can comprise a pump for sucking in an external medium charged with the type of molecule to be detected.

It can also comprise a source of inert gas, which can be nitrogen, positioned downstream of the pump in order to transport the molecules to be detected towards the cavity polymer material.

According to the invention, the sensor can also comprise a removable shutter which makes it possible to separate the cavity polymer material from the fluorescent material.

The means for detecting variation in fluorescence can advantageously comprise a light source for illuminating the fluorescent material and photodetection means for collecting at least a part of the light emitted by the complex formed between the fluorescent material and the molecules to be detected or for measuring the reduction in the light emitted by the “crude” material following the adsorption of the molecule to be detected, that is to say following the formation of the complex.

Another subject matter of the invention is a method for chemical detection of a type of molecule by a sensor according to the invention, characterized in that it comprises the following stages:
the capture by selective adsorption of the type of molecules to be detected by the polymer material comprising “molecularly imprinted” cavities,
the desorption of said molecules by a secondary gas stream after capture by the polymer material,
the formation of a complex between the fluorescent material and the molecules to be detected by movement of the gas stream, charged with molecules to be detected, to the fluorescent material,
the measurement of variation in fluorescence between the fluorescent material and the complex formed.

Advantageously, the capture of the type of molecules to be detected can be carried out with a pump, so as to collect a stream external to the sensor charged with molecules to be detected.

According to the invention, the method can comprise the closing of a shutter which makes it possible to isolate the polymer material comprising cavities from the fluorescent material during the capturing operation. It can then also comprise the opening of the shutter during the desorption operation, so as to send the secondary stream charged with molecules to be detected in the direction of the fluorescent material.

The invention will be better understood on reading the description which will follow, given without implied limitation, and by virtue of the appended figures, among which:

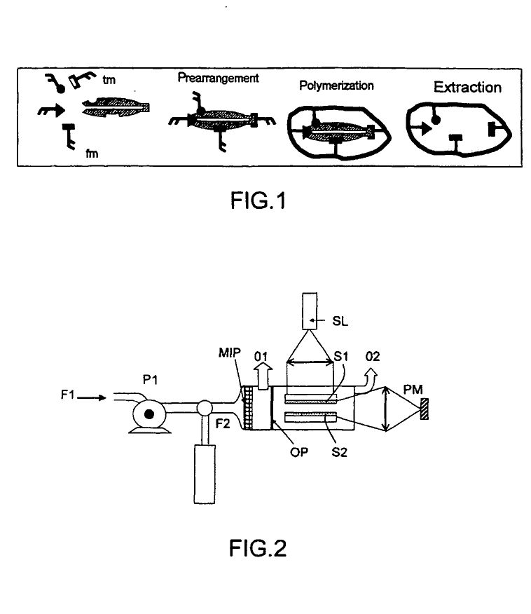

FIG. 1 diagrammatically represents the process for the preparation of molecularly imprinted material,

FIG. 2 illustrates an example of a chemical sensor according to the invention.

Generally, the sensor according to the invention comprises a filter comprising a molecularly imprinted polymer, prepared from the molecule to be detected, carried by a support. The support can be composed either of a functionalized membrane or of an assembly of functionalized microbeads.

Generally, the “molecularly imprinted” polymers (MIPS) are robust biomimetic systems which make it possible to selectively capture a type of given molecule.

Just like biological receptors, MIPs benefit from high affinity and good selectivity for given molecules. A priori, it is possible to imagine MIPs in the image of any functional molecule or family of functional molecules (“molecular meccano”): thus, it is possible to envisage the “tailor-made” synthesis of MIPs and more particularly for target molecules for which no biological equivalent exists.

Due to their highly crosslinked chemical structure, MIPs exhibit very good thermal and chemical stability. Moreover, they have the advantage of being synthesized from inexpensive reactants. The MIPs can have different natures: organic, organic/inorganic hybrid or inorganic. As summarized in the diagram illustrated in FIG. 1 and described in greater detail below, the molecularly imprinted polymer (MIP) is obtained by polymerization, using an initiator and in the presence of a crosslinking agent, of one or more types of polyfunctional monomers (fm) in the presence of a “template” molecule (tm) which can be either directly the molecule to be detected or a steric and chemical analog. During a first “prearrangement” stage, the template molecule develops interactions with one or more functional molecules in a porogenic solvent. During a 2nd “polymerization” stage, the addition of a crosslinking agent and of a polymerization initiator results in the formation of a synthetic matrix encompassing the recognition sites specifically constructed around the template molecule. During the 3rd “extraction” stage, the template molecule is removed using an appropriate solvent: finally, a polymer matrix is obtained exhibiting cavities, referred to as “imprints”, the geometric and chemical configuration of which is perfectly suited to the fixing of the molecules of interest.

By way of example, and in the case of detection of explosives, the MIP can be a hybrid gel obtained from a mixture of silicon alkoxides, such as tetramethoxysilane and methyltrimethoxysilane, some of which can be functionalized by organic groups, for example the following alkoxide:

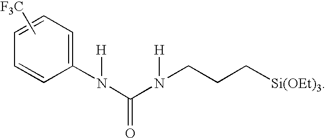

The MIP hybrid gel can subsequently be obtained by reaction of these monomers by hydrolysis and polycondensation in the presence of water and of ethanol (it being possible furthermore for an acid or basic catalyst to be added) and in the presence of the molecule “to be imprinted” (in particular 2,4-DNT, a by-product from the manufacture of TNT, which has a higher vapor pressure than TNT).

The chemical sensor according to the invention thus exhibits an upstream part capable of selectively filtering one type of molecules and a downstream part comprising the fluorescent material and thereby sites for the formation of complexes which are capable of creating variations in fluorescence which are representative of the presence, and even of the concentration, of said molecules in the environment in which the sensor will have been placed.

The fluorescence processes will now be described in more detail and the variations in fluorescence due to the presence of a complex, the physical phenomenon used in the present invention, will now be illustrated.

Generally, the energy transfer between the host material and the molecule to be detected can be described by the following mechanism:

The transfer process takes place in four stages:
1) Absorption of a photon of energy E₀ by the host
2) Relaxation by the environment by a quantity such that the energy available for a radiative transition of the host is E₁<E₀
3) Transfer of the energy E₁ to the dopant/quenching site complex
4) Return to the ground state by a non-radiative process, which explains the reduction in the intensity of fluorescence.

Thus, when a light beam illuminates the fluorescent material at the frequency ν₀ (energy E₀), the radiation of energy E₁ takes place at the frequency ν₁.

In the presence of the fluorescent material/molecule to be detected complex, a portion of the energy E₁ is converted into heat, which brings about a reduction in the intensity scattered by the material.

This variation in amount of energy to be detected by the photodetection means is thus representative of the presence of molecules to be captured.

Moreover, another pathway for reducing the fluorescence is photoinduced electron transfer, which proceeds via oxidation or reduction phenomena after excitation of “donor” or “acceptor” molecules.

Example of Sensor and of Detection Method According to the Invention:

This example is illustrated in FIG. 2:

At the inlet of the sensor, a pump P1 feeds the sensor with an exterior stream F1 of ambient air comprising molecules to be detected. Typically, in the case where explosives are suspected, an attempt will be made to detect traces of 2,4-DNT, which are intrinsic to the presence of TNT.

An upstream chamber is thus formed by closing the shutter OP, so as to isolate the filter from the downstream detection part of the sensor, formed at the fluorescent material.

After a given pumping time (as short as possible: in any case, less than qq minutes), the membrane (MIP) has accumulated enough molecules within its pores to trigger the desorption operation.

Advantageously, but not necessarily, a source of inert gas, typically nitrogen, optionally accompanied by heating means, is positioned at the outlet of the pump P1 in order to generate a stream F2 which desorbs the molecularly imprinted material and makes it possible to generate an inert stream charged with the molecules to be detected which is conveyed towards the downstream part of the sensor by opening the shutter OP. Advantageously, the use of an inert gas makes it possible to limit the photochemical decomposition of the fluorescent polymer.

An opening O1 is provided for releasing, outside the sensor, the inert gas charged with impurities other than the molecules which it is desired specifically to detect.

The stream F2 charged with molecules to be detected is transported to the substrates covered with fluorescent material. The latter can typically be deposited at the surface of two substrates (S1, S2) oriented parallel to the direction of the stream F2, so as to optimize the surface area for exchange between said stream and the sites capable of generating charge transfer complexes in the fluorescent polymer.

A second opening O2 is also provided in the downstream part of the sensor in order to allow the stream F2 to be discharged.

The measurement means comprise a source of light SL of laser or laser diode type which can typically emit in the vicinity of 450 nm for the detection of DNT molecules with the fluorescent polymers described above, which source of light will irradiate all of the substrates carrying fluorescent polymer. A photodetector (PM) of PhotoMultiplier or CCD camera type is placed perpendicular to the source of light so as to collect a part of the radiation scattered by the polymer charged with molecules to be detected without collecting the incident light directly emitted by the source. Typically, in the case of detection of DNT with the fluorescent polymers described above, the photodetector can detect wavelengths centered around 530 nm (representative of the radiation of energy E₁ explained above).

1. A chemical sensor intended for the detection of a type of molecule comprising:
a fluorescent material capable of forming;
a charge transfer complex with the type of molecule to be detected; and
means for measuring the variation in fluorescence of said material, wherein it additionally comprises a filter comprising a polymer material comprising molecularly imprinted cavities, the geometric and chemical configuration of which is defined so as to fix the type of molecule to be detected.

2. The sensor as claimed in claim 1, wherein the fluorescent material is a polymer comprising a π-conjugated chain of the following type

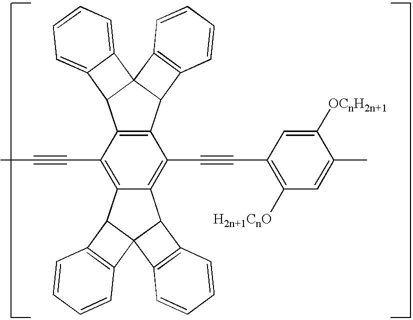

3. The sensor as claimed in claim 1, wherein the fluorescent material is a polymer comprising side chains of the following type:

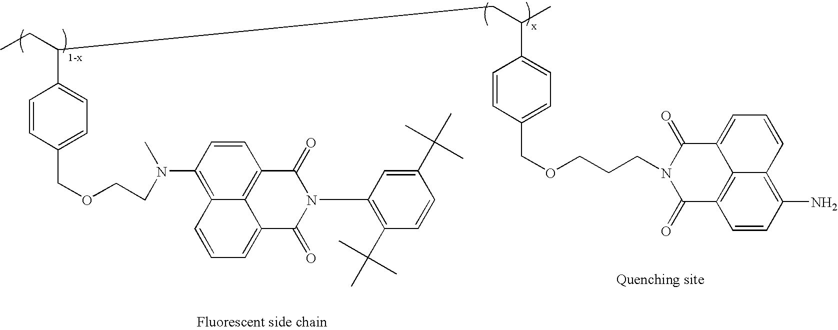

4. The sensor as claimed in claim 1, wherein the polymer material comprising molecularly imprinted cavities is synthesized from functional monomers which make it possible to generate interactions of hydrogen bond type 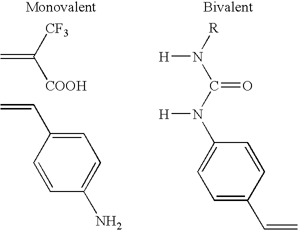
or of π-π interactions type 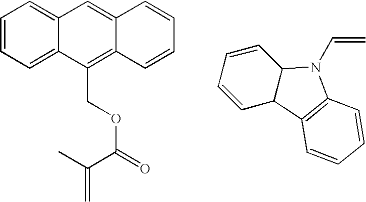
or of metal-ligand complexes type

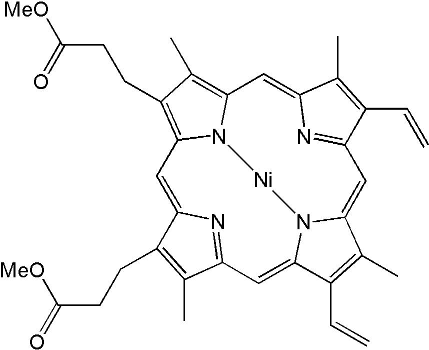

5. The sensor as claimed in claim 1, wherein the fluorescent material is deposited as a thin layer at the surface of at least one first substrate.

6. The sensor as claimed in claim 1, wherein the polymer material comprising molecularly imprinted cavities is formed at the surface of a membrane or at the surface of microbeads held in a porous support positioned perpendicular to the charged stream or positioned parallel to the gas stream and arranged in a column of chromatography column type.

7. The sensor as claimed in claim 1, comprising a pump for sucking in an external medium charged with the type of molecule to be detected.

8. The sensor as claimed in the claim 1, comprising a source of inert gas, which can be nitrogen, positioned downstream of the pump in order to transport the molecules to be detected towards the cavity polymer material.

9. The sensor as claimed in claim 1, comprising a removable shutter which makes it possible to separate the cavity polymer material from the fluorescent material.

10. The sensor as claimed in claim 1, wherein the means for detecting variation in fluorescence comprise a light source for illuminating the fluorescent material and photodetection means for collecting at least a part of the light emitted by the complex formed between the fluorescent material and the molecules to be detected or detecting its reduction therein following the formation of the complex.

11. A method for chemical detection of a type of chemical molecule by a sensor as claimed in claim 1, comprising the following stages steps:
capturing of the type of molecules to be detected by the polymer material comprising molecularly imprinted cavities,
desorption of said molecules by a secondary gas stream after capture by the polymer material,
formation of a complex between the fluorescent material and the molecules to be detected by movement of the gas stream, charged with molecules to be detected, to the fluorescent material,
measurement of variation in fluorescence between the fluorescent material and the complex formed.

12. The method for chemical detection as claimed in claim 11, wherein the capture of the type of molecules to be detected is carried out with a pump, so as to collect a primary stream external to the sensor charged with molecules to be detected.

13. The method for chemical detection as claimed in claim 11, comprising the closing of a shutter which makes it possible to isolate the polymer material comprising cavities from the fluorescent material during the capturing operation.

14. The method for chemical detection as claimed in claim 13, wherein the opening of the shutter during the desorption operation, so as to send the secondary stream charged with molecules to be detected in the direction of the fluorescent material.
